(19) United States
(12) Patent Application Publication
(10) Pub. No.: US 2007/0073385 A1
(43) Pub. Date: Mar. 29, 2007

(54) Eluting, implantable medical device

(75) Inventors: Darin G. Schaeffer, Bloomington, IN (US); David D. Grewe, West Lafayette, IN (US); Patrick H. Ruane, Redwood City, CA (US)

Correspondence Address: BRINKS HOFER GILSON & LIONE/CHICAGO/COOK, PO BOX 10395, CHICAGO, IL, 60610, US

(73) Assignees: Cook Incorporated, Bloomington, IN (US); MED Institute, Inc., West Lafayette, IN (US)

(21) Appl. No.: 11/523,797

(22) Filed: Sep. 18, 2006

Related U.S. Application Data
(60) Provisional application No. 60/718,855, filed on Sep. 20, 2005.

(51) Int. Cl. A61F 2/90

(52) U.S. Cl. 623/1.16; 623/1.42

(57) ABSTRACT

An intraluminal device is provided with a porous structure. The porous structure may be loaded with a bioactive substance to treat surrounding tissues after the intraluminal device has been implanted. The porous structure may be made by depositing a metal film on a foam structure using chemical vapor deposition. Porous structures may also be made by sintering or applying a ceramic layer to the intraluminal device. An intraluminal device is also provided with a ceramic material applied to generally straight portions of the device structure but not to portions adapted to bend. One advantage is that the ceramic material is less likely to fracture since it is applied to regions that experience less strain.

This application claims priority to U.S. Provisional Application No. 60/718,855 filed Sep. 20, 2005 which is hereby incorporated by reference herein.

### BACKGROUND

The present invention relates generally to medical devices and more particularly to intraluminal devices with a porous structure.

A variety of intraluminal devices are known to those in the medical arts, including stents, stent-grafts, filters, occluders, artificial valves and other endoprosthetic devices. For example, stents have now become a relatively common device for treating a number of organs, such as the vascular system, colon, biliary tract, urinary tract, esophagus, trachea and the like. Stents are useful in a variety of medical procedures and are often used to treat blockages, occlusions, narrowing ailments and other related problems that restrict flow through a passageway. Stents are also useful in treating other ailments including various types of aneurysms.

Although stents and other medical devices are used in many different procedures, one common medical procedure in which stents are used involves implanting an endovascular stent into the vascular system. Stents have been shown to be useful in treating numerous vessels throughout the vascular system, including coronary arteries, peripheral arteries (e.g., carotid, brachial, renal, iliac and femoral), and other vessels. However, the use of stents in coronary arteries has drawn particular attention from the medical community because of the growing number of people suffering from heart problems associated with stenosis (i.e., a narrowing of an arterial lumen). This has lead to an increased demand for medical procedures to treat stenosis of the coronary arteries. In addition, the medical community has adapted many intravascular coronary procedures to other intraluminal disorders. The widespread frequency of heart problems may be due to a number of societal changes, including the tendency of people to exercise less while eating greater quantities of unhealthy foods, in conjunction with the fact that people generally now have longer life spans than previous generations. Stents have become a popular alternative for treating coronary stenosis because stenting procedures are considerably less invasive than other alternatives. Traditionally, stenosis of the coronary arteries has been treated with bypass surgery. In general, bypass surgery involves splitting the chest bone to open the chest cavity and grafting a replacement vessel onto the heart to bypass the blocked, or stenosed, artery. However, coronary bypass surgery is a very invasive procedure that is risky and requires a long recovery time for the patient.

Many different types of stents and stenting procedures are possible. In general, however, stents are typically designed as tubular support structures that may be inserted percutaneously and transluminally through a body passageway. Typically, stents are made from a metallic or other synthetic material with a series of radial openings extending through the support structure of the stent to facilitate compression and expansion of the stent. However, other types of stents are designed to have a fixed diameter and are not generally compressible. Although stents may be made from many types of materials, including non-metallic materials, common examples of metallic materials that may be used to make stents include stainless steel, nitinol, cobalt-chrome alloys, amorphous metals, tantalum, platinum, gold and titanium. Typically, stents are implanted within an artery or other passageway by positioning the stent within the lumen to be treated and then expanding the stent from a compressed diameter to an expanded diameter. The ability of the stent to expand from a compressed diameter makes it possible to thread the stent through narrow, tortuous passageways to the area to be treated while the stent is in a relatively small, compressed diameter. Once the stent has been positioned and expanded at the area to be treated, the tubular support structure of the stent contacts and radially supports the inner wall of the passageway. As a result, the implanted stent mechanically prevents the passageway from closing and keeps the passageway open to facilitate fluid flow through the passageway. However, this is only one example of how a stent may be used, and stents may be used for other purposes as well.

Particular stent designs and implantation procedures vary widely. For example, stents are often generally characterized as either balloon-expandable or self-expandable. However, the uses for balloon-expandable and self-expandable stents frequently overlap and procedures related to one type of stent are frequently adapted to other types of stents.

Balloon-expandable stents are frequently used to treat stenosis of the coronary arteries. Usually, balloon-expandable stents are made from ductile materials that plastically deform relatively easily. In the case of stents made from metal, 316L stainless steel which has been annealed is a common choice for this type of stent. One procedure for implanting balloon-expandable stents involves mounting the stent circumferentially on the balloon of a balloon-tipped catheter and threading the catheter through a vessel passageway to the area to be treated. Once the balloon is positioned at the narrowed portion of the vessel to be treated, the balloon is expanded by pumping saline through the catheter to the balloon. The balloon then simultaneously dilates the vessel and radially expands the stent within the dilated portion. The balloon is then deflated and the balloon-tipped catheter is retracted from the passageway. This leaves the expanded stent permanently implanted at the desired location. Ductile metal lends itself to this type of stent since the stent may be compressed by plastic deformation to a small diameter when mounted onto the balloon. When the balloon is later expanded in the vessel, the stent once again plastically deforms to a larger diameter to provide the desired radial support structure. Traditionally, balloon-expandable stents have been more commonly used in coronary vessels than in peripheral vessels because of the deformable nature of these stents. One reason for this is that peripheral vessels tend to experience frequent traumas from external sources (e.g., impacts to a person's arms, legs, etc.) which are transmitted through the body's tissues to the vessel. In the case of peripheral vessels, there is an increased risk that an external trauma could cause a balloon-expandable stent to once again plastically deform in unexpected ways with potentially severe and/or catastrophic results. In the case of coronary vessels, however, this risk is minimal since coronary vessels rarely experience traumas transmitted from external sources. In addition, one advantage of balloon-expandable stents is that the expanded diameter of the stent may be precisely controlled during implantation. This is possible because the pressure applied to the balloon may be controlled by the physician to produce a precise amount of radial expansion and plastic deformation of the stent.

Self-expandable stents are increasingly being used by physicians because of their adaptability to a variety of different conditions and procedures. Self-expandable stents are usually made of shape memory materials or other elastic materials that act like a spring. Typical metals used in this type of stent include nitinol and 304 stainless steel. However, other materials may also be used. A common procedure for implanting self-expandable stents involves a two-step process. First, the narrowed vessel portion to be treated may be dilated with an angioplasty balloon. Second, the stent is implanted into the portion of the vessel that has been dilated. Other variations are also possible, such as adding an additional dilation step after the stent has been implanted or implanting the stent without dilation. To facilitate stent implantation, the stent is normally installed on the end of a catheter in a low profile, compressed state. The stent is typically retained in the compressed state by inserting the stent into a sheath at the end of the catheter. The stent is then guided to the portion of the vessel to be treated. Once the catheter and stent are positioned adjacent the portion to be treated, the stent is released by pulling, or withdrawing, the sheath rearward. Normally, a step or other feature is provided on the catheter to prevent the stent from moving rearward with the sheath. After the stent is released from the retaining sheath, the stent radially springs outward to an expanded diameter until the stent contacts and presses against the vessel wall. Traditionally, self-expandable stents have been used in a number of peripheral arteries in the vascular system due to the shape memory characteristic of these stents. One advantage of self-expandable stents for peripheral arteries is that traumas from external sources do not permanently deform the stent. As a result, the stent may temporarily deform during unusually harsh traumas and spring back to its expanded state once the trauma is relieved. However, self-expandable stents may be used in many other applications as well.

The above-described examples are only some of the applications in which intraluminal devices are used by physicians. Many other applications for intraluminal devices are known and/or will be developed in the future. For example, similar procedures and treatments may also be applicable to vascular filters, occluders, artificial valves and other endoprosthetic devices.

The function of intraluminal devices may be enhanced in certain applications by adding a drug or other bioactive component to the intraluminal device. For example, in the case of stents, one problem that has been encountered with typical stenting procedures is restenosis (i.e., a re-narrowing of the vessel). Restenosis may occur for a variety of reasons, such as the vessel wall collapsing or the growth of new cellular tissue. For example, restenosis may occur as the result of damage caused to the vessel lining during balloon expansion and vessel dilation. This may cause the intima layers of the vessel to attempt to grow new intima tissue to repair the damage. The tendency of vessels to regrow new tissue may be referred to as neointimal hyperplasia. In addition, the synthetic materials that are usually used in stents may also contribute to neointimal hyperplasia. This is caused by the body's tendency to grow new living tissues around and over newly implanted foreign objects. The effect of these responses may result in a re-narrowing of the vessel. However, restenosis is not completely predictable and may occur either abruptly soon after the stenting procedure due to a collapse in the vessel or may occur slowly over a longer period of time for other reasons. In any event, restenosis may defeat the original purpose of the stenting procedure, which is generally to open a narrowed portion of a vessel and to maintain the patency of the vessel.

One approach that has been offered to address the problem of restenosis has been to coat stents with drugs that are designed to inhibit cellular growth. Although many such drugs are known, common examples of these types of drugs include Paclitaxel, Sirolimus and Everolimus. However, despite the benefits of these types of drugs, numerous problems still exist with the way that various drugs and other bioactive substances are combined with stents and other intraluminal devices.

The simplest technique for combining beneficial bioactive substances with an intraluminal device involves coating the bioactive substance directly onto the outer surfaces of the device. Alternatively, various pits or reservoirs may be designed into the intraluminal device to receive the bioactive substance. Common coating processes include dipping, spraying or painting the desired bioactive substance onto the intraluminal device. However, current techniques for combining bioactive substances with intraluminal devices suffer from numerous problems. For example, coatings that are applied to the surfaces of a device may be worn off before the device is implanted. As a result, only a portion of the bioactive substance may remain on the device after implantation to serve the medicinal purpose. This may lead to an ineffective or non-uniform physiological response to the bioactive substance that remains on the device. In addition, it may be desirable for the bioactive substance to be released slowly to the surrounding tissues after implantation so that the effectiveness of the bioactive substance may be maximized. However, it may be difficult to control the release of bioactive substances applied to the outer surfaces of an intraluminal device since the coated surfaces of the device typically come into direct contact with the surrounding tissues or blood flow.

### BRIEF SUMMARY

Intraluminal devices are described with porous structures that may be loaded with a drug or other bioactive substances. One method for making the porous structures includes applying a thin metallic film to a porous foam structure using chemical vapor deposition. Another method includes sintering a metal powder. Additionally, a porous ceramic material may be applied to a substrate using chemical vapor deposition. A method is also described for applying a ceramic layer to regions of a substrate that will experience less strain. Other regions of the substrate that will experience more strain are left uncovered by the ceramic layer to minimize fracturing the ceramic layer. Additional details and advantages are described below in the detailed description.

The invention may include any of the following aspects in various combinations and may also include any other aspect described below in the written description or in the attached drawings.

An expandable stent for medical implantation and elution of a bioactive substance, comprising:
a stent structure formed from a series of structural members, the stent structure being generally cylindrical with an inner surface, an outer surface, a proximal end, and a distal end, wherein a series of radial openings extend through the stent structure between the inner and outer surfaces thereby adapting the stent structure to expand from a compressed diameter to an expanded diameter;
at least a portion of the stent structure being formed from a porous metallic structure, the porous metallic structure having an interconnected, three dimensional network of pores extending therethrough, at least a portion of the pores being open to an exterior surface thereof; and
a bioactive substance loaded into the pores of the porous metallic structure.

The expandable stent, wherein the porous metallic structure comprises at least tantalum.

The expandable stent, wherein the porous metallic structure is greater than 20% porous.

The expandable stent, wherein the bioactive substance is an anti-restenosis drug.

The expandable stent, wherein the porous metallic structure is adjacent a solid metallic substrate.

The expandable stent, wherein the porous metallic structure forms at least a portion of the outer surface of the stent structure.

The expandable stent, wherein the porous metallic structure covers at least two sides of the solid metallic substrate, the porous metallic structure thereby forming at least a portion of the outer surface of the stent structure and at least a portion of the inner surface of the stent structure.

The expandable stent, wherein the porous metallic structure encapsulates at least a portion of the solid metallic substrate.

The expandable stent, wherein the stent structure is formed entirely by the porous metallic structure.

The expandable stent, wherein the porous metallic structure is formed by chemical vapor deposition on a foam structure.

The expandable stent, wherein the porous metallic structure is formed by sintering a metal powder.

The expandable stent, wherein the porous metallic structure is adjacent a solid metallic substrate, the porous metallic structure forming at least a portion of the outer surface of the stent structure, wherein the porous metallic structure comprises at least tantalum, and the bioactive substance is an anti-restenosis drug.

A method of manufacturing an intraluminal device, comprising:
forming a foam structure with an interconnected, three dimensional network of pores extending therethrough, at least a portion of the pores being open to an exterior surface of the foam structure;
depositing a film of metallic material onto the foam structure using chemical vapor deposition, the film infiltrating the foam structure to partially densify the foam structure thereby forming a porous metallic structure;
loading a bioactive substance into the porous metallic structure; and
mounting the porous metallic structure onto a delivery catheter.

The method of manufacturing an intraluminal device, wherein the porous metallic structure comprises at least tantalum.

The method of manufacturing an intraluminal device, wherein the foam structure comprises carbon foam.

The method of manufacturing an intraluminal device, further comprising laser cutting the porous metallic structure before loading the bioactive substance.

The method of manufacturing an intraluminal device, further comprising:
laser cutting a solid metallic substrate;
securing the foam structure to the solid metallic substrate after the laser cutting; and
depositing the film of metallic material after securing the foam structure to the solid metallic substrate.

The method of manufacturing an intraluminal device, further comprising securing the porous metallic structure to a solid metallic substrate after depositing the film of metallic material onto the foam structure.

The method of manufacturing an intraluminal device, further comprising:
securing the foam structure to a solid metallic substrate;
depositing the film of metallic material after securing the foam structure to the solid metallic substrate; and
simultaneously laser cutting the porous metallic structure and the solid metallic substrate after depositing the film of metallic material onto the foam structure.

The method of manufacturing an intraluminal device, wherein the solid metallic substrate is a cannula.

The method of manufacturing an intraluminal device, wherein the foam structure is a cannula.

The method of manufacturing an intraluminal device, wherein the foam structure is a foam cannula, and further comprising:
securing the foam structure to a solid metallic substrate, the solid metallic substrate being a metal cannula that fits inside of the foam cannula;
depositing the film of metallic material after securing the foam structure to the solid metallic substrate; and
simultaneously laser cutting the porous metallic structure and the solid metallic substrate after depositing the film of metallic material onto the foam structure.

The method of manufacturing an intraluminal device, wherein the foam structure comprises carbon foam, the porous metallic structure comprises at least tantalum, and the bioactive substance is an anti-restenosis drug.

A method of treating an intravascular condition, comprising:
accessing a vessel with an introduction catheter;
passing a delivery catheter through the introduction catheter, the delivery catheter comprising an intraluminal device mounted thereon, the intraluminal device comprising a porous metallic structure with an interconnected, three dimensional network of pores extending therethrough, at least a portion of the pores being open to an exterior surface thereof, the pores being loaded with a bioactive substance;
passing the delivery catheter through the vessel to a vessel portion to be treated;
implanting the intraluminal device adjacent the vessel portion; and
withdrawing the delivery catheter from the vessel and the introduction catheter.

The method of treating an intravascular condition, wherein the porous metallic structure comprises at least tantalum.

The method of treating an intravascular condition, wherein the porous metallic structure is greater than 20% porous.

The method of treating an intravascular condition, wherein the bioactive substance is an anti-restenosis drug

The method of treating an intravascular condition, wherein the porous metallic structure is adjacent a solid metallic substrate.

The method of treating an intravascular condition, wherein the porous metallic structure forms at least a portion of an outer surface of the intraluminal device.

The method of treating an intravascular condition, wherein the porous metallic structure covers at least two sides of the solid metallic substrate, the porous metallic structure thereby forming at least a portion of the outer surface of the intraluminal device and at least a portion of an inner surface of the intraluminal device.

The method of treating an intravascular condition, wherein the porous metallic structure encapsulates at least a portion of the solid metallic substrate.

The method of treating an intravascular condition, wherein the intraluminal device is formed entirely by the porous metallic structure.

The method of treating an intravascular condition, wherein the porous metallic structure is formed by chemical vapor deposition on a foam structure.

The method of treating an intravascular condition, wherein the porous metallic structure is formed by sintering a metal powder.

The method of treating an intravascular condition, wherein the porous metallic structure forms at least a portion of an outer surface of the intraluminal device, the porous metallic structure being greater than 20% porous, and wherein the bioactive substance is an anti-restenosis drug.

A method of manufacturing an intraluminal device, comprising:
depositing a layer of ceramic material onto a solid substrate using chemical vapor deposition, the layer having pores extending therethrough;
loading a bioactive substance into the pores; and
mounting the porous metallic structure onto a delivery catheter.

The method of manufacturing an intraluminal device, wherein the ceramic material is aluminum oxide.

The method of manufacturing an intraluminal device, wherein the pores are nanopores.

The method of manufacturing an intraluminal device, wherein the solid substrate is metallic.

The method of manufacturing an intraluminal device, further comprising masking a bended portion of the solid substrate adapted to bend before depositing the layer of ceramic material and leaving a straight portion of the solid substrate unmasked.

The method of manufacturing an intraluminal device, further comprising depositing the layer of ceramic material directly onto a straight portion of the solid substrate without depositing the layer of ceramic material on a bended portion of the solid substrate adapted to bend.

The method of manufacturing an intraluminal device, further comprising:
depositing the layer of ceramic material on a first region of a cannula made from the solid substrate;
leaving a second region of the cannula uncovered by the layer of ceramic material;
cutting an expandable structure from the cannula, the expandable structure comprising first portions adapted to remain generally straight and second portions adapted to bend; and
wherein the first portions are cut from the first region and the second portions are cut from the second region.

The method of manufacturing an intraluminal device, wherein the ceramic material is aluminum oxide, the solid substrate is metallic, and a laser is used to cut the expandable structure.

### BRIEF DESCRIPTION OF SEVERAL VIEWS OF THE DRAWINGS

The invention may be more fully understood by reading the following description in conjunction with the drawings, in which:

FIG. 1 is a plan view of a stent structure, showing the stent in an expanded configuration;

FIG. 2 is a plan view of the stent structure, showing the stent in a collapsed configuration;

FIG. 3 is a cross sectional view of a portion of an intraluminal device, showing the entire cross section being a porous material;

FIG. 4 is a cross sectional view of a portion of an intraluminal device, showing an outer layer of porous material adhered to a solid substrate;

FIG. 5 is a cross sectional view of a portion of an intraluminal device, showing an outer layer of porous material and an inner layer of porous material adhered to a solid substrate;

FIG. 6 is a cross sectional view of a portion of an intraluminal device, showing a solid substrate encapsulated by a layer of porous material;

FIG. 7 is a cross sectional view of a portion of an intraluminal device, showing an outer layer of ceramic material adhered to a solid substrate;

FIG. 8 is a plan view of a stent structure, showing the stent in an expanded configuration, with a layer of ceramic material applied to straight portions of the structure;

FIG. 9 is a plan view of a stent structure, showing the stent in a collapsed configuration, with a layer of ceramic material applied to one region but not applied to another region;

FIG. 10 is a magnified view of a porous metallic structure; and

FIG. 11 is an enlarged, magnified view of the porous metallic structure.

### DETAILED DESCRIPTION

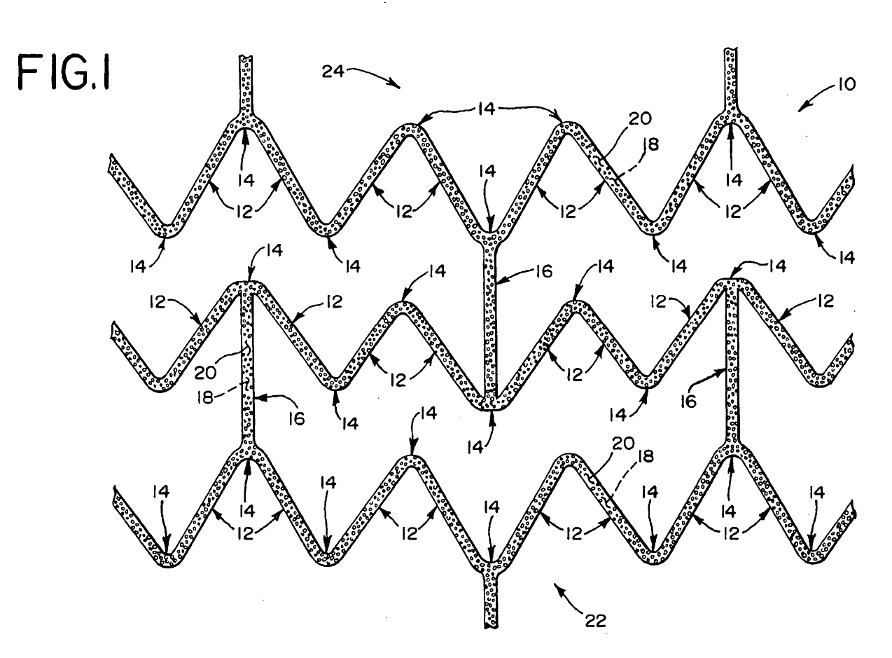

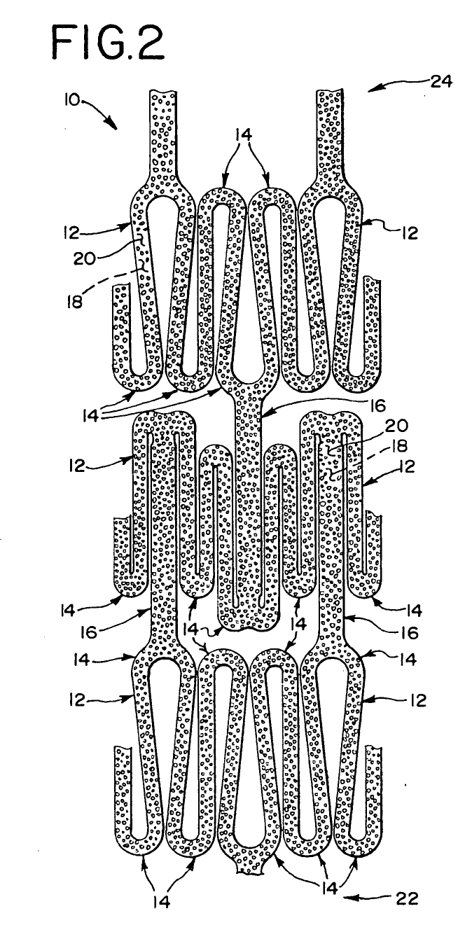

Referring now to the drawings, and particularly to FIGS. 1 and 2, an endoluminal stent 10 is shown. The structure of the stent 10 that is shown is only one example of the type of stent structure that may be used and many other stent structures known in the art may also be used. The stent 10 is made from a series of angular struts 12 interconnected with bends 14. Longitudinal struts 16 may also be used to interconnect the bends 14 and angular struts 12, thereby forming a cylindrical structure with an inner surface 18, an outer surface 20, a proximal end 22 and a distal end 24. Preferably, the stent 10 is expandable between a collapsed configuration as shown in FIG. 2 and an expanded configuration as shown in FIG. 1.

Typically, the collapsed configuration is suitable for introducing the stent 10 into a vessel of a patient and passing the stent 10 through the vessel to a portion to be treated. This may be achieved using a variety of different procedures which may be adapted to particular intraluminal devices. For example, the stent 10 may be mounted on the distal end of a delivery catheter. Where the stent 10 is a balloon-expandable stent, the stent 10 may be mounted on a balloon which contacts the inner surface 18 of the stent 10. Where the stent 10 is a self-expandable stent, the stent 10 may be mounted within a retaining sheath which contacts the outer surface 20 of the stent 10 and retains the stent 10 in the collapsed configuration. A patient's vessel may then be accessed using techniques that are well known to medical professionals. For example, a hollow needle may be used to penetrate the vessel, and a guide wire may be threaded through the needle into the vessel. The needle may then be removed and replaced with an introduction catheter. The introduction catheter generally serves the purpose of being a port which provides access to the vessel and through which various intraluminal tools and devices may be passed. The delivery catheter with the stent 10 mounted thereon may then be passed through the introduction catheter and through the vessel to a vessel portion to be treated.

Once the stent 10 is positioned adjacent the vessel portion to be treated, the stent 10 is implanted by either expanding the balloon or retracting the restraining sheath. This causes the stent 10 to expand to its expanded configuration as shown in FIG. 1 so that the outer surface 20 of the stent 10 contacts the vessel wall. The delivery catheter may than be withdrawn from the vessel and the introduction catheter. These techniques are not limited to stents, however, and may also be applicable to other intraluminal devices, such as vascular filters, occluders, artificial valves and other endoprosthetic devices.

One advantage of the stent 10 is that at least a portion of the stent 10 includes a porous material which may have several benefits. One porous material that may be used is a porous metal known as Trabecular Metal sold by Zimmer, Inc. This material has also been known as Hedrocel and sold by Implex Corporation. Descriptions of this type of material may be found in U.S. Pat. Nos. 5,282,861; 6,063,442; and 6,087,553, each of which is incorporated herein by reference. In general porous metals of this type may be made by forming a foam structure out of carbon or other materials so that the foam structure has pores extending through the structure in three dimensions. The pores are further open to the exterior surface of the foam structure. The foam structure is then partially densified with a metallic material using chemical vapor deposition (CVD). The metallic material infiltrates the foam structure and deposits a thin film on the structure of the foam without completely filling in the pores. As a result, a porous metallic structure is formed. In the porous metallic structure, an interconnected, three dimensional network of pores are formed that extend through the structure. At least a portion of the pores are open to an exterior surface so that drugs or other bioactive substances may be loaded into the porous structure and released at a treatment site. Examples of a porous metallic structure that may be made using the process described above are shown in FIGS. 10 and 11. In FIG. 10, the porous metallic structure is shown at a magnification of about 12×. FIG. 11 is an enlarged view of the porous metallic structure. As shown, the porous metallic structure is formed from a foam-like metallic structure 60 with pores 62 extending therethrough in an interconnected, three dimensional network. Although various metal materials may be used, tantalum and tantalum alloys are preferred since porous structures using these materials are currently available. Moreover, tantalum has been shown to be highly biocompatible. In addition, tantalum is highly radiopaque.

However, other processes for making a porous metal structure may be used. For example, the porous metallic structure may also be made by sintering. Sintering involves filling a mold with a metal powder and applying pressure to compact the metal powder. The molded metal structure is then heated below the melting point of the metal to form metallurgical bonds between the metal particles. Because the metal is not fully melted, the process does not result in a solid metal structure. Instead, pores remain in the structure between the individual metal particles of the metal powder. As a result, pores extend through the structure in three dimensions with the pores being open to the exterior surface of the structure. Other processes for forming a porous metallic structure may also be possible.

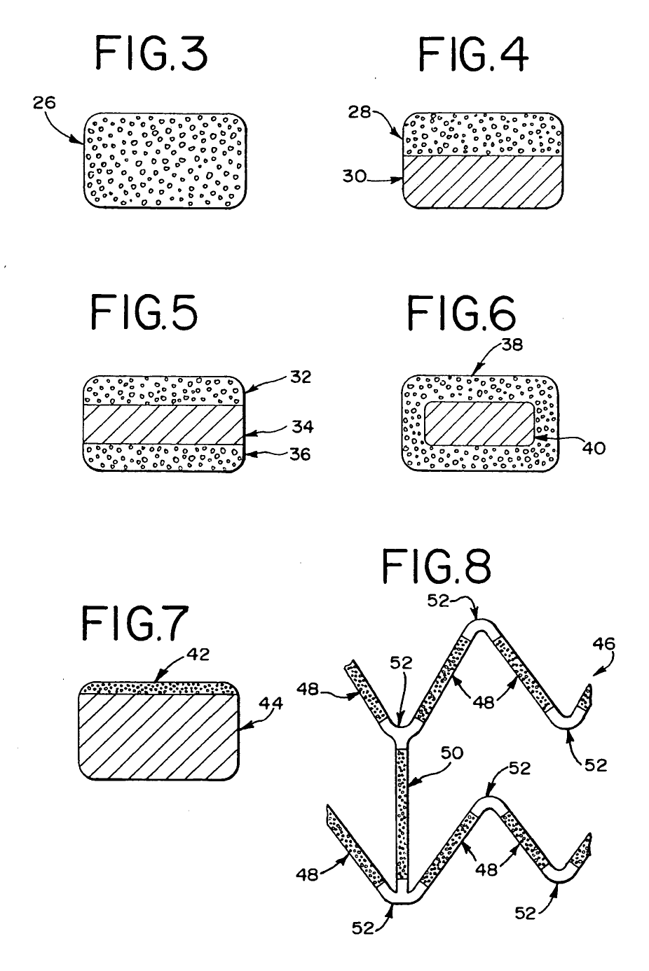

FIGS. 3 through 6 show cross sectional views of some structures that are possible for intraluminal devices using porous metallic structures. For example, the cross sectional views may represent the angular struts 12, bends 14 or longitudinal struts 16 of the stent 10 shown in FIGS. 1 and 2. However, porous metallic structures may also be used in other intraluminal devices as well. In addition, the entire structure of the intraluminal device may be made as shown or only a part of the structure may be made with the porous metallic structure. In FIG. 3, the full cross section 26 is formed from a porous metallic structure. This type of structure may be constructed either by CVD, sintering or other processes. For example, in the case of a stent, a cannula may be made from a porous metallic material. The cannula may then be cut with a laser to form angular struts 12, bends 14 and longitudinal struts 16 as described above. In FIG. 4, an outer layer 28 of porous metallic material is adhered to a solid metallic substrate 30. This type of structure may be constructed using a CVD process by securing a foam structure to a solid metallic substrate and then depositing a metal film on the structure using a CVD process. In the case of a stent, the solid metallic substrate and the foam structure may both be cannulas with the foam cannula being secured to the outer diameter of the substrate cannula. Various methods of securing structures to each other may be used including gluing, clamping, welding or heating. Alternatively, the porous metallic structure may be made separately from the solid metallic substrate using a CVD, sintering or other process. The porous metallic structure and the solid metallic substrate may then be secured together after the porous metallic structure has been made. The combined structure may then be laser cut to form the final structure or other subsequent processing steps may be performed. In FIG. 5, an outer layer 32 of porous metallic material is adhered to the outside of a solid metallic substrate 34, and an inner layer 36 of porous metallic material is adhered to the inside of the solid metallic substrate 34. This type of structure may be constructed using CVD, sintering or other processes as described above. In FIG. 6, a layer 38 of porous metallic material encapsulates a solid metallic substrate 40. Although this structure may be constructed in various ways, it is preferred to secure a foam structure completely around a solid metallic substrate that has already been shaped by laser cutting or the like. A CVD process may then be used to deposit a metal film throughout the foam structure to form a porous metallic structure. If desired, a second laser cutting step may then be used to shape the porous metallic structure along the sides of the solid metallic substrate.

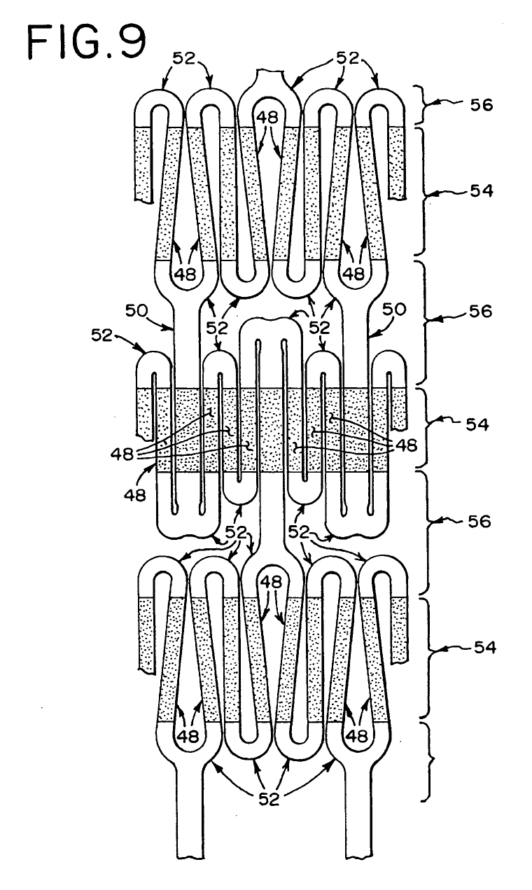

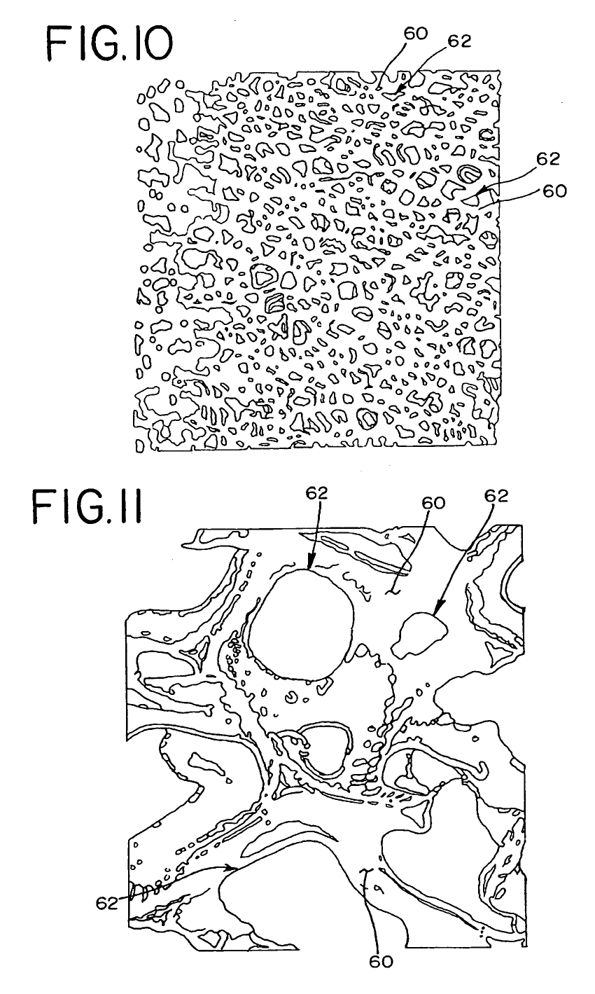

Porous structures may also be made from other materials and other processes as well. For example, metal oxides or ceramic materials may be adhered to a solid substrate using CVD or other processes. In FIG. 7, another cross sectional view of a structure for an intraluminal device is shown. As with FIGS. 3 through 6, this structure may be used in a variety of intraluminal devices including stents. In FIG. 7, a thin layer 42 of aluminum oxide is applied to a solid metallic substrate 44 using a CVD process. Because ceramic materials exhibit a brittle character when applied in thick layers, a thin layer that will minimize fracturing of the ceramic layer is preferred. The thin, ceramic layer 42 is porous and may have particularly small nanopores. In addition, as shown in FIG. 8, the ceramic layer 42 may be applied only to portions of the intraluminal device structure where bending strains are expected to be minimal. This may allow the thickness of the ceramic layer to be thicker while still minimizing fractures. For example, in the case of an expandable stent 46, the ceramic layer 42 may be applied along the straight portions of the angular struts 48 and the longitudinal struts 50. To avoid cracking or fracturing of the ceramic layer 42, the ceramic layer 42 is not applied to the bends 52 of the stent structure since the bends 52 typically experience high levels of bending strain. This type of structure may be constructed in a variety of ways. For example, the bends 52 of a pre-cut structure may be masked by an agent that is impervious to CVD. After the ceramic layer is applied using a CVD process, the masking agent may be removed from the bends 52, thereby preventing the application of a ceramic layer to the bends 52. The ceramic layer 42 may also be precisely applied directly to the straight portions of the angular struts 48 and the longitudinal struts 50 without masking the bends 52. Alternatively, as shown in FIG. 9, regions or bands 54 of a ceramic layer may be applied to a metallic cannula or other substrate material. The bands 54 may be separated by regions 56 without a ceramic layer. A stent structure or other intraluminal device structure may then be laser cut from the substrate material so that only generally straight sections 48, 50 are cut from the regions 54 with the bands of ceramic layer. The bends 52 may then be cut from the regions 56 without a ceramic layer.

Although a wide variety of bioactive substances may be used with the structures and devices described herein, a few examples are as follows.

Anti-angiogenic bioactive materials include any protein, peptide, chemical, or other molecule which acts to inhibit vascular growth. A variety of methods may be readily utilized to determine whether a given bioactive material has anti-angiogenic activity, including for example, chick chorioallantoic membrane (“CAM”) assays. Briefly, a portion of the shell from a freshly fertilized chicken egg is removed, and a methyl cellulose disk containing a sample of the anti-angiogenic bioactive material to be tested is placed on the membrane. After several days (e.g., 48 hours), inhibition of vascular growth by the sample to be tested may be readily determined by visualization of the chick chorioallantoic membrane in the region surrounding the methyl cellulose disk. Inhibition of vascular growth may also be determined quantitatively, for example, by determining the number and size of blood vessels surrounding the methyl cellulose disk, as compared to a control methyl cellulose disk. Although anti-angiogenic bioactive materials as described herein are considered to inhibit the formation of new blood vessels if they do so in merely a statistically significant manner, as compared to a control, within preferred aspects such anti-angiogenic bioactive materials completely inhibits the formation of new blood vessels, as well as reduce the size and number of previously existing vessels. In addition to the CAM assay described above, a variety of other assays may also be utilized to determine the efficacy of anti-angiogenic bioactive materials in vivo, including for example, mouse models which have been developed for this purpose (see Roberston et al., Cancer. Res. 51:1339-1344, 1991).

A wide variety of anti-angiogenic bioactive materials may be coated on or within an implantable medical device. Representative examples include compounds which disrupt microtubule function, Anti-Invasive Factors, retinoic acid and derivatives thereof, Suramin, Tissue Inhibitor of Metalloproteinase-1, Tissue Inhibitor of Metalloproteinase-2, Plasminogen Activator Inhibitor-1, Plasminogen Activator Inhibitor-2, and various forms of the lighter “d group” transition metals. These and other anti-angiogenic bioactive materials will be discussed in more detail below.

Representative examples of anti-angiogenic therapeutic agents which disrupt microtubule function include estramustine (available from Sigma; Wang and Stearns Cancer Res. 48:6262-6271, 1988), epothilone, curacin-A, colchicine, methotrexate, and paclitaxel, vinblastine, vincristine, D20 and 4-tert-butyl-[3-(2-chloroethyl) ureido] benzene (“tBCEU”). Briefly, such compounds can act in several different manners. For example, compounds such as colchicine and vinblastine act by depolymerizing micotubules.

One preferred anti-angiogenic therapeutic agent useful in mitigating or preventing restenosis is paclitaxel, a compound which disrupts microtubule formation by binding to tubulin to form abnormal mitotic spindles. Briefly, paclitaxel is a highly derivatized diterpenoid (Wani et al., J. Am. Chem. Soc. 93:2325, 1971) which has been obtained from the harvested and dried bark of Taxus brevifolia (Pacific Yew.) and Taxomyces Andreanae and Endophytic Fungus of the Pacific Yew (Stierle et al., Science 60:214-216, 1993). “Paclitaxel” (which should be understood herein to include prodrugs, analogues and derivatives such as, for example, TAXOL®, TAXOTERE®, 10-desacetyl analogues of paclitaxel and 3′N-desbenzoyl-3′N-t-butoxy carbonyl analogues of paclitaxel) may be readily prepared utilizing techniques known to those skilled in the art (see WO 94/07882, WO 94/07881, WO 94/07880, WO 94/07876, WO 93/23555, WO 93/10076, WO94/00156, WO 93/24476, EP 590267, WO 94/20089; U.S. Pat. Nos. 5,294,637, 5,283,253, 5,279,949, 5,274,137, 5,202,448, 5,200,534, 5,229,529, 5,254,580, 5,412,092, 5,395,850, 5,380,751, 5,350,866, 4,857,653, 5,272,171, 5,411,984, 5,248,796, 5,248,796, 5,422,364, 5,300,638, 5,294,637, 5,362,831, 5,440,056, 4,814,470, 5,278,324, 5,352,805, 5,411,984, 5,059,699, 4,942,184; Tetrahedron Letters 35(52):9709-9712, 1994; J. Med. Chem. 35:4230-4237, 1992; J. Med. Chem. 34:992-998, 1991; J. Natural Prod; 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).

Representative examples of such paclitaxel derivatives or analogues include 7-deoxy-docetaxol, 7,8-Cyclopropataxanes, N-Substituted 2-Azetidones, 6,7-Epoxy Paclitaxels, 6,7-Modified Paclitaxels, 10-Desacetoxytaxol, 10-Deacetyltaxol (from 10-deacetylbaccatin III), Phosphonooxy and Carbonate Derivatives of Taxol, Taxol 2′,7-di(sodium 1,2-benzenedicarboxylate, 10-desacetoxy-11,12-dihydrotaxol-10,12(18)-diene derivatives, 10-desacetoxytaxol, Protaxol (2′-and/or 7-O-ester derivatives), (2′- and/or 7-O-carbonate derivatives), Asymmetric Synthesis of Taxol Side Chain, Fluoro Taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′—OH-7-PEG(5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl) taxol; 2′-[2-(N,N-dimethylamino)propionyl]taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N, N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin).

Briefly, Anti-Invasive Factor material, or “AIF” which is prepared from extracts of cartilage, contains constituents which are responsible for inhibiting the growth of new blood vessels. These constituents comprise a family of 7 low molecular weight proteins (<50,000 daltons) (Kuettner and Pauli, “Inhibition of neovascularization by a cartilage factor” in Development of the Vascular System, Pitman Books (CIBA Foundation Symposium 100), pp. 163-173, 1983), including a variety of proteins which have inhibitory effects against a variety of proteases (Eisentein et al, Am. J. Pathol. 81:337-346, 1975; Langer et al., Science 193:70-72, 1976; and Horton et al., Science 199:1342-1345, 1978). AIF suitable for use within the present invention may be readily prepared utilizing techniques known in the art (e.g., Eisentein et al, supra; Kuettner and Pauli, supra; and Langer et al., supra). Purified constituents of AIF such as Cartilage-Derived Inhibitor (“CDI”) (see Moses et al., Science 248:1408-1410, 1990) may also be readily prepared and utilized within the context of the present invention.

Retinoic acids alter the metabolism of extracellular matrix components, resulting in the inhibition of angiogenesis. Addition of proline analogs, angiostatic steroids, or heparin may be utilized in order to synergistically increase the anti-angiogenic effect of transretinoic acid. Retinoic acid, as well as derivatives thereof which may also be utilized in the context of the present invention, may be readily obtained from commercial sources, including for example, Sigma Chemical Co. (#R2625).

Suramin is a polysulfonated naphthylurea compound that is typically used as a trypanocidal agent. Briefly, Suramin blocks the specific cell surface binding of various growth factors such as platelet derived growth factor (“PDGF”), epidermal growth factor (“EGF”), transforming growth factor (“TGF-β”), insulin-like growth factor (“IGF-1”), and fibroblast growth factor (“βFGF”). Suramin may be prepared in accordance with known techniques, or readily obtained from a variety of commercial sources, including for example Mobay Chemical Co., New York. (see Gagliardi et al., Cancer Res. 52:5073-5075, 1992; and Coffey, Jr., et al., J. of Cell. Phys. 132:143-148, 1987).

Tissue Inhibitor of Metalloproteinases-1 (“TIMP”) is secreted by endothelial cells which also secrete MMPases. TIMP is glycosylated and has a molecular weight of 28.5 kDa. TIMP-1 regulates angiogenesis by binding to activated metalloproteinases, thereby suppressing the invasion of blood vessels into the extracellular matrix. Tissue Inhibitor of Metalloproteinases-2 (“TIMP-2”) may also be utilized to inhibit angiogenesis. Briefly, TIMP-2 is a 21 kDa nonglycosylated protein which binds to metalloproteinases in both the active and latent, proenzyme forms. Both TIMP-1 and TIMP-2 may be obtained from commercial sources such as Synergen, Boulder, Colo.

Plasminogen Activator Inhibitor-1 (PA) is a 50 kDa glycoprotein which is present in blood platelets, and can also be synthesized by endothelial cells and muscle cells. PAI-1 inhibits t-PA and urokinase plasminogen activator at the basolateral site of the endothelium, and additionally regulates the fibrinolysis process. Plasminogen Activator Inhibitor-2 (PAI-2) is generally found only in the blood under certain circumstances such as in pregnancy, and in the presence of tumors. Briefly, PAI-2 is a 56 kDa protein which is secreted by monocytes and macrophages. It is believed to regulate fibrinolytic activity, and in particular inhibits urokinase plasminogen activator and tissue plasminogen activator, thereby preventing fibrinolysis.

Other therapeutic agents which may be utilized within the present invention include lighter “d group” transition metals, such as, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate (i.e., VO3−) and orthovanadate (i.e., VP43−) complexes such as, for example, ammonium metavanadate (i.e., NH4VO3), sodium metavanadate (i.e., NaVO3), and sodium orthovanadate (i.e., Na3VO4). Suitable vanadyl (i.e., VO2+) complexes include, for example, vanadyl acetylacetonate and vanadyl sulfate including vanadyl sulfate hydrates such as vanadyl sulfate mono- and trihydrates, Bis[maltolato(oxovanadium)] (IV)] (“BMOV”), Bis[(ethylmaltolato)oxovanadium] (IV) (“BEOV”), and Bis(cysteine, amide N-octyl)oxovanadium (IV) (“naglivan”).

Representative examples of tungsten and molybdenum complexes also include oxo complexes. Suitable oxo tungsten complexes include tungstate and tungsten oxide complexes. Suitable tungstate,(i.e., WO42−) complexes include ammonium tungstate (i.e., (NH4)2WO4), calcium tungstate (i.e., CaWO4), sodium tungstate dihydrate (i.e., Na2WO4.2H2O), and tungstic acid (i.e., H2WO4). Suitable tungsten oxides include tungsten (IV) oxide (i.e., WO2) and tungsten (VI) oxide (i.e., WO3). Suitable oxo molybdenum complexes include molybdate, molybdenum oxide, and molybdenyl complexes. Suitable molybdate (i.e., MoO42−) complexes include ammonium molybdate (i.e., (NH4)2MoO4) and its hydrates, sodium molybdate (i.e., Na2MoO4) and its hydrates, and potassium molybdate (i.e., K2MoO4) and its hydrates. Suitable molybdenum oxides include molybdenum (VI) oxide (i.e., MoO2), molybdenum (VI) oxide (i.e., MoO3), and molybdic acid. Suitable molybdenyl (i.e., MoO22+) complexes include, for example, molybdenyl acetylacetonate. Other suitable tungsten and molybdenum complexes include hydroxo derivatives derived from, for example, glycerol, tartaric acid, and sugars.

Other anti-angiogenic bioactive materials include Platelet Bioactive material 4 (Sigma Chemical Co., #F1385); Protamine Sulphate: (Clupeine) (Sigma Chemical Co., #P4505); Sulphated Chitin Derivatives (prepared from queen crab shells), (Sigma Chemical Co., #C3641; Murata et al., Cancer Res. 51:22-26, 1991); Sulphated Polysaccharide Peptidoglycan Complex (SP-PG) (the function of this compound may be enhanced by the presence of steroids such as estrogen, and tamoxifen citrate); Staurosporine (Sigma Chemical Co., #S4400); Modulators of Matrix Metabolism, including for example, proline analogs {[(L-azetidine-2-carboxylic acid (LACA) (Sigma Chemical Co., #A0760)), cishydroxyproline, d,L-3,4-dehydroproline (Sigma Chemical Co., #D0265), Thiaproline (Sigma Chemical Co., #T0631)], α,α-dipyridyl (Sigma Chemical Co., #D7505), β-aminopropionitrile fumarate (Sigma Chemical Co., #A3134)]}; MDL 27032 (4-propyl-5-(4-pyridinyl)-2(3H)-oxazolone; Merion Merrel Dow Research Institute); Methotrexate (Sigma Chemical Co., #A6770; Hirata et al., Arthritis and Rheumatism 32:1065-1073, 1989); Mitoxantrone (Polverini and Novak, Biochem. Biophys. Res. Comm. 140:901-907); Heparin (Folkman, Bio. Phar. 34:905-909, 1985; Sigma Chemical Co., #P8754); Interferons (e.g., Sigma Chemical Co., #13265); 2 Macroglobulin-serum (Sigma Chemical Co., #M7151); ChIMP-3 (Pavloff et al., J. Bio. Chem. 267:17321-17326, 1992); Chymostatin (Sigma Chemical Co., #C7268; Tomkinson et al., Biochem J. 286:475-480, 1992); β-Cyclodextrin Tetradecasulfate (Sigma Chemical Co., #C4767); Eponemycin; Camptothecin; Fumagillin and derivatives (Sigma Chemical Co., #F6771; Canadian Patent No. 2,024,306; Ingber et al., Nature 348:555-557, 1990); Gold Sodium Thiomalate (“GST”; Sigma, G4022; Matsubara and Ziff, J. Clin. Invest. 79:1440-1446, 1987); (D-Penicillamine (“CDPT”; Sigma Chemical Co., #P4875 or P5000(HCl)); β-1-anticollagenase-serum; α2-antiplasmin (Sigma Chem. Co.:A0914; Holmes et al., J. Biol. Chem. 262(4):1659-1664, 1987); Bisantrene (National Cancer Institute); Lobenzarit disodium (N-(2)-carboxyphenyl-4-chloroanthronilic acid disodium or “CCA”; Takeuchi et al., Agents Actions 36:312-316, 1992); Thalidomide; Angostatic steroid; AGM-1470; carboxynaminolmidazole; and metalloproteinase inhibitors such as BB94, estrogen and estrogen analogues, antiestrogens, antioxidants, bioflavonoids (Pycnogenol), ether lipids (s-phosphonate, ET-18-OCH3), tyrosine kinase inhibitors (genisteine, erbstatin, herbamycin A, lavendustine-c, hydroxycinnamates), α chemokines [Human interferon-inducible protein 10 (IP-10)], —C—X—C— Chemokines (Gro-beta), Nitric Oxide, Antifungal Agents (Radicicol), 15-deoxyspergualin, Metal Complexes (Titanocene dichloride-cyclopentadienyl titanium dichloride), Triphenylmethane Derivatives (aurintricarboxylic acid), Linomide, Thalidomide, IL-12, Heparinase, Angiostatin, Antimicrobial Agents (Minocycline), Plasma Proteins (Apolipoprotein E), Anthracyclines (TAN-1120), Proliferin-Related Protein, FR-111142, Saponin of Panax ginseng (Ginsenoside-Rb2), and Pentosan polysulfate.

An “antineoplastic bioactive” is an agent that inhibits or prevents the growth and spread of neoplasms or malignant cells. Therapeutic agents with antineoplastic properties include, for example, heparin, covalent heparin, aspirin, colchicine, a retinoid, an antisense nucleotide, cyclosporine, dexamethasone, dexamethasone sodium phosphate, dexamethasone acetate, or another dexamethasone derivative, angiopeptin, ascorbic acid, alpha tocopherol, estrogen or another sex hormone, AZT or other antipolyermases, finasteride (Proscar®), ritonavir (Norvir®), sirolimus, tacrolimus, everolimus, ABT-578, a mammalian target of rapamycin (mTOR), monoclonal antibodies capable of blocking smooth muscle cell proliferation, thymidine kinase inhibitors, and epothilones.

Other antineoplastic agents may generally be classified according to five categories: (1) aklylating agents; (2) antimetabolites; (3) antineoplastic antibiotics; and (4) hormones and antihormones; and (5) natural antineoplastic products.

Suitable alkylating agents include, for example, amifostine, busulfan, carboplatin, mustine, mustine hydrochloride, carmustine, chlorambucil, cisplatin, cyclophosphamide, cyclophosphamide monohydrate, anhydrous cyclophosphamide, mafosfamide, trofosfamide, trilophosphamide, trophosphamide, dacarbazine, ifosfamide, lomustine, mechlorethamine, mechlorethamine hydrochloride, melphalan, mesna, pipobroman, streptozocin, triethylenethiophosphaoramide (thio-TEPA), and uracil mustard.

Suitable antimetabolites include, for example, cladribine, cytarabine, floxuridine, fludarabine phosphate, fluoropyrimidine prodrugs, fluorouracil, 5-fluorouracil, hydroxyurea, levamisole hydrochloride, mercaptopurine, mercaptopurine monohydrate, purinethiol or anhydrous mercaptopurine, thioguanine, anyhydrous thioguanine, thiguanine hemihydrate, tioguanine, azathioprine, azathioprine sodium, methotrexate, methotrexate sodium, methotrexate disodium, teniposide and other epipodophyllotoxins, and thioguanine.

Suitable antineoplastic antibiotics include, for example, bleomycin, bleomycin sulfate, actinomycins, such as actinomycin-D and actinomycin-C, dactinomycin, meractinomycin, daunorubicins, such as daunorubicin hydrochloride, daunomycin hydrochloride, or rubidomycin hydrochloride, doxorubicin, epirubicin, epirubicin hydrochloride, idarubicin, idarubicin hydrochloride, priarubicin, tepirubicin, zorubicin, zorubicin hydrochloride, menogaril, mitozantrone, mitozantrone hydrochloride, mitomycin, mitotane, mitoxantrone hydrochloride, piroxantrone, prixantrone hydrochloride, antrhapyrazole hydrochloride, oxantrazole hydrochloride, pentostatine, and plicamycin.

Suitable hormones and antihormones include, for example, anastrozole, bicalutamide, estramustine phosphate sodium, flutamide, goserelin acetate, irinotecan hydrochloride and other camptothecins such as topotecan hydrochloride, leuprolide acetate, nilutamide, tamoxifen, tamoxifen citrate, and vinca alkaloids such as vinblastine, vinblastine sulfate, vincaleukoblastine sulphate, vincristine, vincristine suflate, vinorelbine, vinorelbine tartrate, vinorelbine ditartrate, vindesine, vindesine sulfate, desacetyl vinblastine amide sulfate.

Suitable natural antineoplastics include asparaginase, taxanes such as docetaxel and paclitaxel and derivatives thereof, and interferons, such as interferon alfa-2a, recombinant interferon alfa-2a, and interferon alfa-2b.

One benefit of the structures described above is that the porous structures may be loaded with drugs or other bioactive substances. For example, in the case of stents, anti-restenosis drugs like Paclitaxel, Sirolimus and Everolimus May have desirable physiological effects. Depending on the particular treatment, it may be desirable to load the pores of the porous structures with other bioactive substances or a combination of different bioactive substances. For example, bioactive substances that encourage specific tissue growth or promote healing of the surrounding tissues may be desirable. The porosity of the porous structures may also be useful in encouraging cellular migration into the pores of the intraluminal device. This may result in the intraluminal device being incorporated into the tissue structure.

One advantage of loading bioactive substances into the porous structures is that the pores may tend to retain the bioactive substance more securely and thereby release the bioactive substance more slowly over time. This may increase the length of time in which the bioactive substance effectively treats the tissues. Moreover, the porous structures may have a larger capacity to store a greater quantity of a bioactive substance compared with conventional coatings. The loaded bioactive substances may also be less susceptible of being worn off the intraluminal device since the bioactive substance is stored within the pores instead of directly on the outer surface of the device. This may result in a more reliable treatment by the bioactive substance since the quantity of the bioactive substance that is actually delivered to the tissues being treated may be more predictable. In addition, the bioactive substance or other substance which is loaded into the pores may make the surface of the porous structure more lubricious. This may be helpful in the case of self-expandable stents where the porous structure is adhered to the outer surface of the stent. In this case, friction between the restraining sheath and the outer surface of the stent may be reduced, thereby making it easier to precisely release the stent from the restraining sheath.

A method of manufacturing an intraluminal device is provided comprising: forming a foam structure with an interconnected, three dimensional network of pores extending therethrough, at least a portion of said pores being open to an exterior surface of said foam structure; depositing a film of metallic material onto said foam structure using chemical vapor deposition, said film infiltrating said foam structure to partially densify said foam structure thereby forming a porous metallic structure; loading a bioactive substance into said porous metallic structure; and mounting said porous metallic structure onto a delivery catheter.

Other aspects of the above-described method may include any combination of the following features. The method wherein said porous metallic structure comprises at least tantalum. The method wherein said foam structure comprises carbon foam. The method further comprising laser cutting said porous metallic structure before loading said bioactive substance. The method further comprising: laser cutting a solid metallic substrate; securing said foam structure to said solid metallic substrate after said laser cutting; and depositing said film of metallic material after securing said foam structure to said solid metallic substrate. The method further comprising securing said porous metallic structure to a solid metallic substrate after depositing said film of metallic material onto said foam structure. The method further comprising: securing said foam structure to a solid metallic substrate; depositing said film of metallic material after securing said foam structure to said solid metallic substrate; and simultaneously laser cutting said porous metallic structure and said solid metallic substrate after depositing said film of metallic material onto said foam structure. The method wherein said solid metallic substrate is a cannula. The method wherein said foam structure is a cannula. The method wherein said foam structure is a foam cannula, and further comprising: securing said foam structure to a solid metallic substrate, said solid metallic substrate being a metal cannula that fits inside of said foam cannula; depositing said film of metallic material after securing said foam structure to said solid metallic substrate; and simultaneously laser cutting said porous metallic structure and said solid metallic substrate after depositing said film of metallic material onto said foam structure. The method wherein said foam structure comprises carbon foam, said porous metallic structure comprises at least tantalum, and said bioactive substance is an anti-restenosis drug.

A method of treating an intravascular condition is provided comprising: accessing a vessel with an introduction catheter; passing a delivery catheter through said introduction catheter, said delivery catheter comprising an intraluminal device mounted thereon, said intraluminal device comprising a porous metallic structure with an interconnected, three dimensional network of pores extending therethrough, at least a portion of said pores being open to an exterior surface thereof, said pores being loaded with a bioactive substance; passing said delivery catheter through said vessel to a vessel portion to be treated; implanting said intraluminal device adjacent said vessel portion; and withdrawing said delivery catheter from said vessel and said introduction catheter.

Other aspects of the above-described method may include any combination of the following features. The method wherein said porous metallic structure comprises at least tantalum. The method wherein said porous metallic structure is greater than 20% porous. The method wherein said bioactive substance is an anti-restenosis drug. The method wherein said porous metallic structure is adjacent a solid metallic substrate. The method wherein said porous metallic structure forms at least a portion of an outer surface of said intraluminal device. The method wherein said porous metallic structure covers at least two sides of said solid metallic substrate, said porous metallic structure thereby forming at least a portion of said outer surface of said intraluminal device and at least a portion of an inner surface of said intraluminal device. The method wherein said porous metallic structure encapsulates at least a portion of said solid metallic substrate. The method wherein said intraluminal device is formed entirely by said porous metallic structure. The method wherein said porous metallic structure is formed by chemical vapor deposition on a foam structure. The method wherein said porous metallic structure is formed by sintering a metal powder. The method wherein said porous metallic structure forms at least a portion of an outer surface of said intraluminal device, said porous metallic structure being greater than 20% porous, and wherein said bioactive substance is an anti-restenosis drug.

A method of manufacturing an intraluminal device is provided comprising: depositing a layer of ceramic material onto a solid substrate using chemical vapor deposition, said layer having pores extending therethrough; loading a bioactive substance into said pores; and mounting said porous metallic structure onto a delivery catheter.

Other aspects of the above-described method may include any combination of the following features. The method wherein said ceramic material is aluminum oxide. The method wherein said pores are nanopores. The method wherein said solid substrate is metallic. The method further comprising masking a bended portion of said solid substrate adapted to bend before depositing said layer of ceramic material and leaving a straight portion of said solid substrate unmasked. The method further comprising depositing said layer of ceramic material directly onto a straight portion of said solid substrate without depositing said layer of ceramic material on a bended portion of said solid substrate adapted to bend. The method further comprising: depositing said layer of ceramic material on a first region of a cannula made from said solid substrate; leaving a second region of said cannula uncovered by said layer of ceramic material; cutting an expandable structure from said cannula, said expandable structure comprising first portions adapted to remain generally straight and second portions adapted to bend; and wherein said first portions are cut from said first region and said second portions are cut from said second region. The method wherein said ceramic material is aluminum oxide, said solid substrate is metallic, and a laser is used to cut said expandable structure.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

We claim:

1. An expandable stent for medical implantation and elution of a bioactive substance, comprising:
a stent structure formed from a series of structural members, said stent structure being generally cylindrical with an inner surface, an outer surface, a proximal end, and a distal end, wherein a series of radial openings extend through said stent structure between said inner and outer surfaces thereby adapting said stent structure to expand from a compressed diameter to an expanded diameter;
at least a portion of said stent structure being formed from a porous metallic structure, said porous metallic structure having an interconnected, three dimensional network of pores extending therethrough, at least a portion of said pores being open to an exterior surface thereof; and
a bioactive substance loaded into said pores of said porous metallic structure.

2. The expandable stent according to claim 1, wherein said porous metallic structure comprises at least tantalum.

3. The expandable stent according to claim 1, wherein said porous metallic structure is greater than 20% porous.

4. The expandable stent according to claim 1, wherein said bioactive substance is an anti-restenosis drug.

5. The expandable stent according to claim 1, wherein said porous metallic structure is adjacent a solid metallic substrate.

6. The expandable stent according to claim 5, wherein said porous metallic structure forms at least a portion of said outer surface of said stent structure.

7. The expandable stent according to claim 6, wherein said porous metallic structure covers at least two sides of said solid metallic substrate, said porous metallic structure thereby forming at least a portion of said outer surface of said stent structure and at least a portion of said inner surface of said stent structure.

8. The expandable stent according to claim 7, wherein said porous metallic structure encapsulates at least a portion of said solid metallic substrate.

9. The expandable stent according to claim 1, wherein said stent structure is formed entirely by said porous metallic structure.

10. The expandable stent according to claim 1, wherein said porous metallic structure is formed by chemical vapor deposition on a foam structure.

11. The expandable stent according to claim 1, wherein said porous metallic structure is formed by sintering a metal powder.

12. The expandable stent according to claim 1, wherein said porous metallic structure is adjacent a solid metallic substrate, said porous metallic structure forming at least a portion of said outer surface of said stent structure, wherein said porous metallic structure comprises at least tantalum, and said bioactive substance is an anti-restenosis drug.

13. The expandable stent according to claim 1, wherein said porous metallic structure comprises at least tantalum, wherein said porous metallic structure is greater than 20% porous, wherein said bioactive substance is an anti-restenosis drug, wherein said porous metallic structure is adjacent a solid metallic substrate, wherein said porous metallic structure forms at least a portion of said outer surface of said stent structure, wherein said porous metallic structure covers at least two sides of said solid metallic substrate, said porous metallic structure thereby forming at least a portion of said outer surface of said stent structure and at least a portion of said inner surface of said stent structure, wherein said porous metallic structure encapsulates at least a portion of said solid metallic substrate, wherein a portion of said stent structure is formed entirely by said porous metallic structure, wherein said porous metallic structure is formed by chemical vapor deposition on a foam structure or by sintering a metal powder.

14. A method of manufacturing an intraluminal device, comprising:
forming a foam structure with an interconnected, three dimensional network of pores extending therethrough, at least a portion of said pores being open to an exterior surface of said foam structure;
depositing a film of metallic material onto said foam structure using chemical vapor deposition, said film infiltrating said foam structure to partially densify said foam structure thereby forming a porous metallic structure;
loading a bioactive substance into said porous metallic structure; and
mounting said porous metallic structure onto a delivery catheter.

15. A method of treating an intravascular condition, comprising:
accessing a vessel with an introduction catheter;
passing a delivery catheter through said introduction catheter, said delivery catheter comprising an intraluminal device mounted thereon, said intraluminal device comprising a porous metallic structure with an interconnected, three dimensional network of pores extending therethrough, at least a portion of said pores being open to an exterior surface thereof, said pores being loaded with a bioactive substance;
passing said delivery catheter through said vessel to a vessel portion to be treated;
implanting said intraluminal device adjacent said vessel portion; and
withdrawing said delivery catheter from said vessel and said introduction catheter.
